(19) United States
(12) Patent Application Publication
(10) Pub. No.: US 2006/0270014 A1
(43) Pub. Date: Nov. 30, 2006

(54) Thrombin purification

(76) Inventors: Dan Pawlak, Waunakee, WI (US); Bradley H. Knoll, Verona, WI (US); Abdel Hak Terrab, Madison, WI (US); Gerald Chesmore, Milton, WI (US)

Correspondence Address: KING PHARMACEUTICALS, INC., 400 CROSSING BOULEVARD, BRIDGEWATER, NJ, 08807, US

(21) Appl. No.: 11/140,374

(22) Filed: May 26, 2005

(51) Int. Cl. C12N 9/74

(52) U.S. Cl. 435/214

(57) ABSTRACT

This invention relates generally to methods for the preparation of thrombin having a high degree of purity and high specific activity. More specifically, the invention encompasses steps to exclude high molecular weight impurities from thrombin preparations by size exclusion filtration. In additional embodiments, the preparation of thrombin additionally includes an ion exchange filtration step. The methods of the invention are particularly suited for large scale purification of thrombin. The invention also relates to thrombin compositions with reduced levels of high molecular weight impurities. In particular, the levels of factor Va, prions and/or viral agents are greatly reduced.

### 1. FIELD OF THE INVENTION

This invention relates generally to methods for the preparation of thrombin having a high degree of purity and high specific activity. More specifically, the invention encompasses methods comprising excluding high molecular weight impurities from thrombin preparations. The invention also encompasses methods for the preparation of thrombin comprising excluding viral agents from thrombin preparations. The invention also relates to purified thrombin, substantially free of impurities that can contribute to adverse effects in patients caused by large molecular weight impurities such as Factor Va, prions and/or viral agents.

### 2. BACKGROUND OF THE INVENTION

Thrombin is a protolytic enzyme, which appears in the blood following activation of the coagulation system as a result of proteolysis of prothrombin. Thrombin facilitates the clotting of blood by catalyzing the conversion of fibrinogen to fibrin, which forms blood clots, and releases fibrinopeptides A and B. Following a disturbance to the vascular system, the production of thrombin is central to the coagulation process.

Thrombin preparations have been approved by the FDA to be applied topically as an aid to homeostasis whenever oozing blood or minor bleeding from capillaries and small venules is accessible. Topical application of commercially available thrombin significantly speeds coagulation of the blood and significantly reduces clot times.

Studies using low purity thrombin formulations indicate that coagulopathies may occur in patients in response to exposure to low purity, topical thrombin formulations. Impurities typically present in commercially available thrombin preparations include factor Va, bovine serum albumin (BSA), and other high molecular weight proteins. Factor Va contamination of commercial bovine thrombin formulations can stimulate the production of patient anti-bovine factor Va antibodies, which can cross-react with the patient's own factor Va, thereby leading to impaired hemostasis.

The blood clotting strength of thrombin is measured in units per ml. The more concentrated the sample is, the greater the potency, the faster it will coagulate blood (or create fibrinogen). Specific activity is a ratio of the potency of a sample divided by its protein content and is expressed in units per milligram of protein.

Thrombin specific activity is dependent upon the purity of the thrombin. Highly purified thrombin shows an increase in specific activity when compared with a less pure preparation.

Previously, purification of thrombin has been generally limited to the use of conventional ion exchange chromatography. U.S. Pat. No. 5,397,704 describes a bovine/veal preparation of thrombin that is prepared using a series of anion and cation exchange chromatography.

U.S. Pat. No. 5,151,355 describes a bovine thrombin preparation that is made by reacting one unit of prothrombin with less than 5 units of thromboplastin in the presence of calcium. The thrombin thereby obtained is then applied sequentially to an anion-exchange agarose column and a cation exchange agarose chromatography column.

U.S. Pat. No. 4,965,203 discloses a method of purifying bovine thrombin in which the thrombin is passed through a series of ion-exchange chromatography columns and then formulated with a polyol and buffers. Although the above thrombin preparations are alleged to have high specific activity, such purification schemes do not provide any means for effectively eliminating high molecular weight impurities.

US Patent Application Publication 2001/0033837 discloses a method of purifying a thrombin preparation using hydrophobic interaction chromatography, optionally followed by cation exchange chromatography. Although the thrombin prepared by the disclosed method use hydrophobic interaction chromatography, the method described for purification and virus removal are not capable of achieving the virus removal and specific activity or purity encompassed by the instant invention.

Accordingly, there is a need in the art for methods that can be used to produce higher purity thrombin. Preferably, the higher purity thrombin will have lower levels of high molecular weight impurities, including factor Va, and a high clearance margin of viral agents and prions.

3. BRIEF SUMMARY OF THE INVENTION

The present invention is directed to a thrombin composition and method of making a thrombin preparation. In one embodiment the present invention comprises a method for preparing a thrombin having enhanced purity, the method comprising applying the thrombin preparation to a size exclusion filter; and excluding impurities from the thrombin preparation. In the present invention, the thrombin preparation can be bovine thrombin or Thrombin-JMI®.

In addition, the size exclusion filter is capable of excluding impurities that have a molecular weight greater than 40 kDa. Preferably, the size exclusion filter is capable of excluding impurities that have molecular weights ranging from 40 kDa to 300 kDa. More preferably, the size exclusion filter has a molecular weight cutoff ranging from 50 kDa and 150 kDa. In one embodiment the size exclusion filter has a molecular weight cutoff is 50 kDa. In another preferred embodiment the size exclusion filter has a molecular weight cutoff of 100 kDa.

In yet another embodiment of the present invention, the method is capable of reducing impurities in the thrombin preparation by at least 50%, as compared to Thrombin-JMI®, or purified thrombin. More preferably, the method is capable of reducing impurities in the thrombin preparation by at least 80% as compared to pre-purified or low purity bovine thrombin as described in paragraphs 0065-0071.

Another aspect of the present invention the method is capable of increasing the specific activity of the thrombin preparation by at least 1000%, 1200% or 1500%.

In another embodiment the thrombin made by the present invention has enhanced purity and is substantially free of impurities. Preferably, the thrombin preparation made by the present invention has enhanced purity and is either substantially free of impurities or is substantially free of viral agents or both. Also, the thrombin preparation, having enhanced purity, made by the present method is substantially free of factor Va and prions. Additionally, the thrombin preparation, having enhanced purity, made by the present method has a prion reduction equal to at least 3.5 logs. In yet another embodiment the thrombin preparation made by the present invention is substantially pure.

The method of the present invention may also include applying the thrombin preparation to an ion exchange filter. In another embodiment of the present invention, the method further comprises applying the thrombin preparation to a further chromatographic purification step. Preferable the chromatographic purification step comprises an ion exchange chromatography column.

In another embodiment of the method of the present invention the method of preparing a thrombin having enhanced purity comprises applying a thrombin preparation to an ion exchange filter.

In another embodiment of the method of the present invention the method of preparing a thrombin having enhanced purity comprises applying a heat treatment to a thrombin preparation. Preferably the heat treatment includes holding the thrombin at 60° C. for 10 hours.

In another embodiment of the method of the present invention, the method of preparing a thrombin having enhanced purity comprising lowering the pH of a thrombin preparation to about 5 or lower.

In another embodiment of the method of the present invention the method of preparing a thrombin having enhanced purity comprises the application of electromagnetic radiation to a thrombin preparation. The electromagnetic radiation can be gamma radiation or UV radiation.

The present invention so includes, method for preparing a thrombin having enhanced purity, the method comprising applying the thrombin preparation to a chromatographic purification step; applying the thrombin preparation to a size exclusion filter; applying the thrombin preparation to an ion exchange filter; and excluding impurities from the thrombin preparation. In a preferred embodiment the chromatographic purification step comprises an ion exchange chromatography column or a size exclusion chromatography column.

The present invention is directed to a method for large-scale preparation of thrombin having enhanced purity comprising applying at least 15 L of a thrombin preparation to a size exclusion filter. In a preferred embodiment, the present invention is directed to a method for large-scale preparation of thrombin having enhanced purity comprising applying at least 15 L of a thrombin preparation to a size exclusion filter wherein the 15 L of thrombin preparation comprises about 300,000,000 units of thrombin.

The present invention is also directed to a thrombin composition. In one embodiment the thrombin composition is substantially free of impurities. In another embodiment the thrombin preparation is substantially free of impurities having a molecular weight greater than 40 kDa. Preferably, the thrombin composition is substantially free of impurities having a molecular weight between 40 kDa and 300 kDa.

In yet another embodiment the thrombin composition of the present invention is substantially pure. Preferably, the thrombin composition substantially free of factor Va. More preferably, the factor Va is present at less than 0.4 μg/1000 units of thrombin. Additionally, the amount of factor Va can be measured by factor Va activity assay, ELISA, or Western Blot.

The present invention is also directed to a thrombin composition substantially free of viral agents.

In another embodiment of the present invention, the thrombin composition has specific activity greater than 1800 u/mg of protein and is substantially free of impurities having a molecular weight greater than 40 kDa. The thrombin composition of the present invention can have a specific activity between about 1800 and 3000 u/mg of protein. Preferably, the thrombin composition can have a specific activity between about 2400 and 2500 u/mg of protein or between about 2500 and 2600 u/mg of protein, between about 2600 and 2700 u/mg of protein, between about 2700 and 2800 u/mg of protein, between about 2800 and 2900 u/mg of protein or between about 2900 and 3000 u/mg of protein. Additionally, the thrombin composition can have a specific activity greater than 3000 u/mg of protein. The thrombin composition of the present invention can be substantially free of viral agents, wherein the log reduction value is greater than 3.5 per virus.

### 4. BRIEF DESCRIPTION OF THE DRAWINGS

FIG. 1 shows a flow diagram of all of the steps used to prepare a thrombin preparation, in accordance with the method of the present invention.

FIG. 2 shows a Sodium Dodecyl Sulfate Polyacrylamide Gel Electrophoresis (SDS-PAGE) comparison of Thrombin-JMI® after the addition of the purification process of the present invention (lanes 7, 8 and 9) to Thrombin-JMI® as currently manufactured (lanes 4 and 5) and the retentive of the size exclusion filtration, showing the high molecular weight impurities (lane 11).

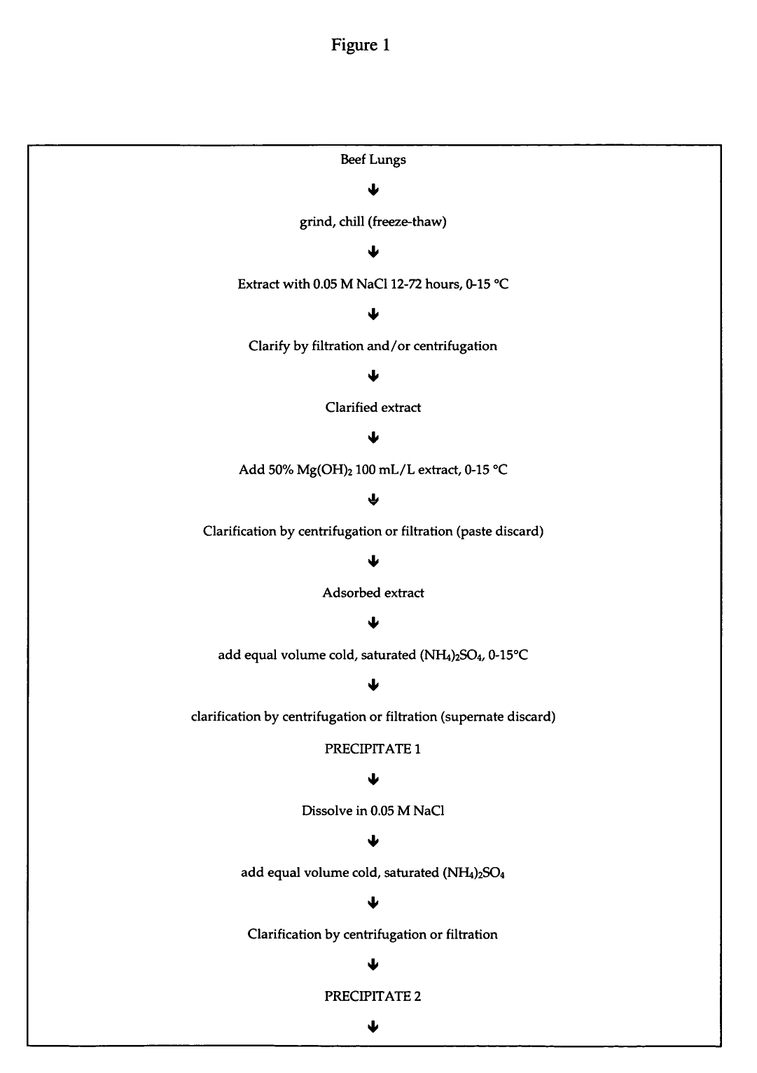

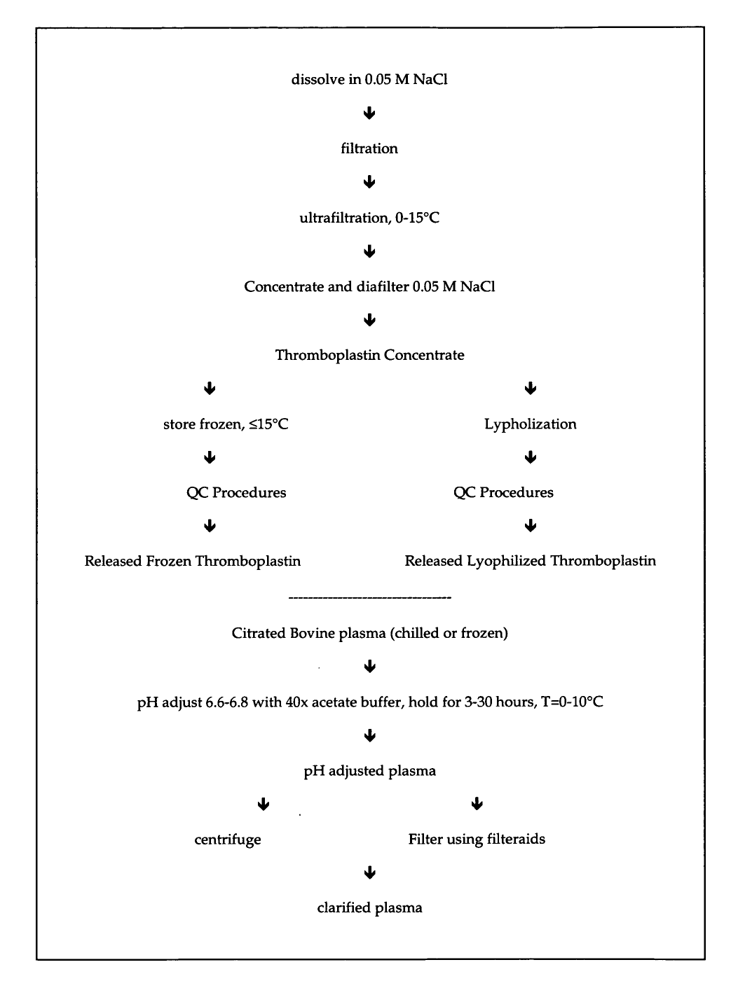

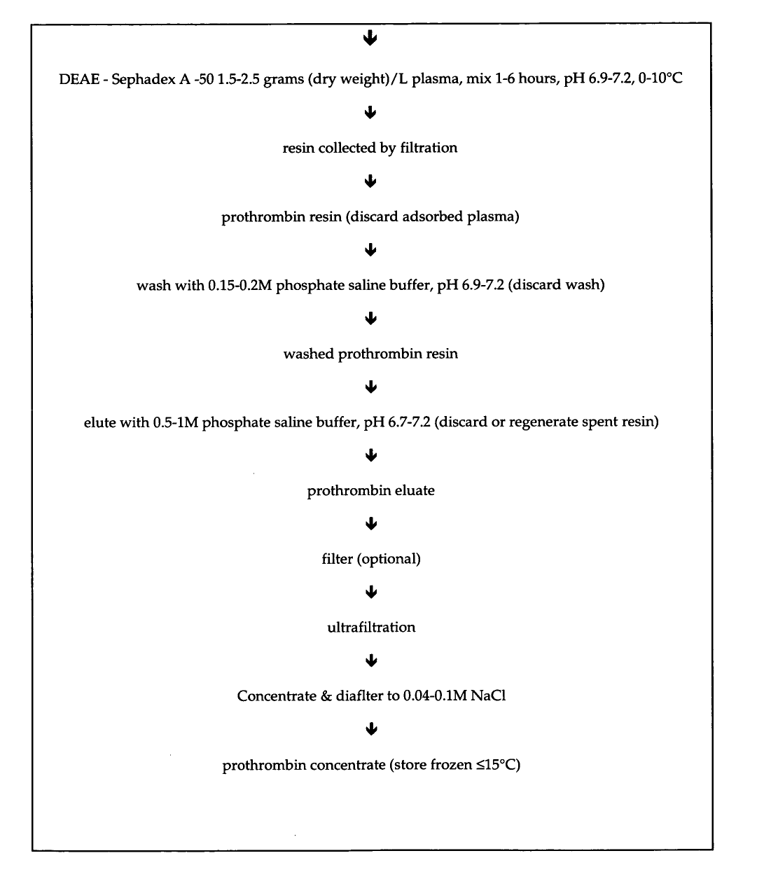

### 5. DETAILED DESCRIPTION OF THE INVENTION

The present invention is based on the discovery that the use of size exclusion filtration for the purification of thrombin provides substantial benefits over prior art methods for the purification of thrombin. Size exclusion filtration produces thrombin that is significantly more pure and safe, due to the elimination of high molecular weight impurities such as Factor Va. Size exclusion filtration also provides a high degree of viral clearance and substantial removal of high molecular weight impurities, along with consistency, reliability, and ease of use.

Also, the use of ion exchange filtration also provides substantial benefits to thrombin purification. Ion exchange filters provide a high degree of viral clearance along with consistency, reliability and ease of use. Other methods of thrombin purification will also be presented below.

This invention encompasses methods for the preparation of thrombin with increased purity, increased specific activity, and increased safety. This invention encompasses applying purifying steps to a thrombin preparation. These steps include, but are not limited to, chromatographic purification; applying the thrombin preparation to a size exclusion filter; applying the thrombin preparation to an ion exchange filter; lowering the pH; or irradiating the thrombin preparation with electromagnetic radiation. These steps may be applied independently or in combination.

Furthermore, the methods are amenable to large scale, commercial production and purification. The methods of the present invention yield thrombin substantially free of impurities, having a molecular weight of greater than 40 kDa, and viral agents. Thus, the methods of the invention provide a great advantage because the purified thrombin is free of high molecular weight impurities which can contribute to adverse effects in patients.

The invention also relates to high purity preparations of thrombin. The invention encompasses thrombin compositions that have a specific activity greater than 1800 u/mg of protein. The invention also encompasses a thrombin composition substantially free of high molecular weight impurities, including, factor Va, bacterial agents, prions and viral agents. Preferably, the thrombin composition has a specific activity greater than 1800 u/mg of protein and is substantially free of high molecular weight impurities.

Specific activity of thrombin can be measured by standard assays known in the art, including clotting assays and chromogenic assays (See, e.g., Gaffney et al., 1995, Thromb Haemost 74:900-903). Factor Va levels can be measured by methods, including, but not limited to gel electrophoresis, factor Va activity assays and antibody based assays.

According to the methods of the present invention, recovery and purification of thrombin preparations can be achieved by excluding impurities using methods involving separation based on molecular weight, preferably size exclusion filtration. Preferably, the filter used is a tangential flow filter. In preferred embodiments, the methods of the invention comprise applying a thrombin preparation to a size exclusion filter capable of excluding impurities that have a molecular weight greater than 40 kDa in size from said thrombin preparation. In a preferred embodiment, the size exclusion filter is capable of excluding impurities that have a molecular weight ranging from 40 kDa to 300 kDa.

In general, any method involving separation based on molecular weight can be used, including size exclusion chromatography and size exclusion filtration. In a preferred embodiment, exclusion of higher molecular weight molecules comprises the use of size exclusion filtration. Preferably, filter pores are large enough to allow the passage of the thrombin molecules, but small enough to retain many impurities, including large protein impurities and viruses. Preferably, a size exclusion filter suitable for the present invention effectively reduces bacterial agents and endotoxins.

Thrombin has a molecular weight of approximately 40 kDa. Thus, size exclusion filters of molecular weight cutoffs of 50, 100, 150, 300 kDa or greater can be used. In one embodiment, the size exclusion filter has a molecular weight cutoff ranging from 40 kDa to 300 kDa. In another embodiment, the size exclusion filter has a molecular weight cutoff ranging from 50 kDa to 150 kDa. In a preferred embodiment, the size exclusion filter has a molecular weight cutoff of 50 kDa. In a more preferred embodiment, the size exclusion filter has a molecular weight cutoff of 100 kDa. This step can also optionally include the application of dia-filtration to maximize thrombin recovery.

In preferred embodiments, the size exclusion filters will have pore sizes with a molecular weight cut off, i.e., exclusion limit, of around 100 kDa. In certain embodiments, these filters are made of modified polyethersulfone on a highly porous polyolefin backing. One example of a filter that can be used in this invention is the Omega™ 100K VR manufactured by PALL FILTRON Corporation.

Other size exclusion filters that may be used in accordance with this invention include, but are not limited to the Viresolve/70 manufactured by Millipore Corporation; VirA/Gard 500 manufactured by A/G Technology, Corporation, and Ultipor DV20 manufactured by Pall Corporation.

With size exclusion filtration the large molecules, including viral impurities, get retained by the pores in the membrane. The membrane can be discarded after use or, in the alternative, the membrane can be reused.

Membranes that result in a high enough log reduction are considered acceptable, and can be used. Each log reduction is a reduction of 90%. Other tests can also be performed on the filter to ensure the filter has an acceptable pore size range.

The resulting thrombin preferably has higher specific activity and lower impurity levels, including factor Va, prions, and viral particle impurities. In certain methods of the invention, viral agents and/or high molecular weight impurities are reduced by at least 50%, at least 60%, at least 75%, at least 80%, at least 85%, at least 90%, at least 95%, or at least 99%. In a preferred embodiment, high molecular weight impurities and/or viral particle impurities in the thrombin preparation are reduced by at least 80%. In certain embodiments, the recovery of thrombin is also greater than 80%, greater than 85%, greater than 90%, or greater than 95%.

The invention also provides methods for obtaining thrombin compositions of increased purity. In certain embodiments, the purity of a thrombin preparation is increased by more than 30%, more than 50%, more than 75%, or more than 90%, as compared to Thrombin-JMI®, or other purified thrombin. In certain embodiments, increased purity is determined by specific activity. In one embodiment, the methods of the invention provide for thrombin preparations where the specific activity of the thrombin preparation is increased by at least 1000%, at least 1200%, at least 1500%, or at least 1800%, as compared pre-purified thrombin. The resulting thrombin preferably has higher specific activity and lower impurity levels, including factor Va, prions and viral particle impurities.

After this filtration step, the specific activity rises from ≧about 1500 u/mg to ≧about 2300 u/mg. Also, factors Va, as well as, other protein impurities are markedly reduced in concentration.

The methods of the invention can be applicable to thrombin preparations isolated from bovine or human sources, and natural or recombinant preparations.

In certain embodiments, methods of the present invention may further comprise applying the thrombin to an ion exchange filter. Ion exchange filtration is a separation method which filters solutes based on their electronic charge. Ion exchange filters contain charge centers on the ion exchange membrane. When the sample is passed through the filter, the charged compounds in the sample will adsorb onto the charge centers on the membrane. A filter is selected that has a positive charge and that will filter out charged protein and viral impurities from the thrombin preparation.

Ion exchange filters used in accordance with this invention are preferably charged positively, whereas ion exchange chromatography resins typically used for thrombin purification are charged negatively. Ion exchange filters are efficient for removing nucleic acids. In a preferred embodiment, an ion exchange filer has pendant quaternary amine groups. A preferred ion exchange filter that can be used with this invention is the Mustang™ Q filter manufactured by the Pall Corporation. Another ion exchange filter that can be used is the Cuno Zeta Plus VRO5.

Other methods to inactivate or remove viruses can be utilized as well. Such methods can be used in addition to the above described methods. These include chromatographic purification, the application of heat, lowering the pH, and the application of gamma and UV irradiation. Moderate heat treatment is an acceptable virus inactivation method. For example, heat treatment for 10 hours at 60° C. can be used.

Chromatographic purification, including but not limited to, ion exchange chromatography, is also an acceptable viral clearance step. Viruses with the same net charge as the ion exchanger will not bind to the resin and will be cleared in the breakthrough.

Lowering the pH, to below about 5 or below about 4 is also an effective virus inactivation procedure. However, some loss of thrombin activity is observed with this method.

Gamma irradiation is a powerful and robust virus inactivation method. Reportedly, gamma irradiation is effective against a wide variety of viruses. However, less than 70% recovery can be obtained in the case of commercially available thrombin. UV light is also an effective virus inactivation procedure. However, some loss of thrombin activity is observed with this method. Short exposure periods may result in less of a loss of activity.

In preferred embodiments, a combination of filtration and viral clearance steps, as described above, are used. In a more preferred embodiment, size exclusion filtration occurs, then subsequently, one or more filtration or viral clearance steps (i.e., ion exchange filtration, ion exchange chromatography, heat treatment, lowering the pH, or irradiation) are used.

The invention encompasses methods comprising applying at least 300 ml of a thrombin preparation to a size exclusion filter. The invention also encompasses methods for large scale, commercial purification of thrombin comprising applying at least 40 L of a thrombin preparation to a size exclusion filter, preferably at least 60 L of a thrombin preparation to a size exclusion filter, most preferably at least 90 L of a thrombin preparation to a size exclusion filter. In certain embodiments, the volume of thrombin preparation applied to the size exclusion filter depends on the surface area of the filters used and/or the number of filters used.

In certain embodiments of the invention, initial volumes of 50 mls, 100 mls, 150 mls, 200 mls, 250 mls, 300 mls, 350 mls, 400 mls, 450 mls, 500 mls, multiples thereof, or more are applied to a size exclusion filter. The methods of the invention are particularly suited to large-scale purifications of thrombin. As such, in preferred embodiments of invention, initial volumes of 15 L to 20 L and multiples thereof are applied to a size exclusion filter. In one embodiment when 15 L of thrombin preparation is applied to the size exclusion filter, the thrombin preparation comprises 300,000,000 units of thrombin.

In other preferred embodiments of the invention, initial volumes of 40 L to 60 L, 60 L to 80 L, 80 L to 100 L, over 100 L, or more and multiples thereof are applied to a size exclusion filter.

The present invention also encompasses thrombin compositions having a specific activity ranging from about 1800 u/mg and 3000 u/mg, more preferably between about 1800 u/mg and 2400 u/mg. In other embodiments, the specific activity is between about 2400 u/mg and 2500 u/mg, between about 2500 u/mg and 2600 u/mg, or between about 2600 u/mg and 2700 u/mg, between about 2700 and 2800 u/mg of protein, between about 2800 and 2900 u/mg of protein or between about 2900 and 3000 u/mg of protein. In certain embodiments, the thrombin has a specific activity greater than 3000 u/mg.

The invention also encompasses thrombin compositions substantially free of high molecular weight impurities. As used herein, a composition that is “substantially free” of a high molecular weight impurity, e.g., factor Va, prions or viral agents, means that the composition contains less than about 5-20% by weight, preferably less than about 15% by weight, more preferably less than about 10% by weight. As used herein, a composition that is “substantially pure” contains less than 5% of the high molecular weight impurities by weight, and most preferably less than about 3% by weight of the high molecular weight impurities.

In certain embodiments of the invention, after performing the size exclusion filtration, factor Va can barely be detected in the concentrated sample (before it is diluted into a final formulation) and is typically not detected at all in the final formulation. In some embodiments, factor Va is reduced by at least 50%, at least 55%, at least 60%, at least 65%, at least 70%, at least 75%, at least 80%, at least 85%, at least 90%, at least 95%, or at least 99%.

The present invention provides methods for purifying thrombin comprising excluding molecules having a higher molecular weight than thrombin. Methods for purifying thrombin to eliminate at least 50%, at least 55%, at least 60%, at least 65%, at least 70%, at least 75%, at least 80%, at least 85%, at least 90%, at least 95% and at least 99% of higher molecular weight impurities are provided. Elimination of higher molecular weight impurities will be based on the particular molecular weight cutoff used, as described above. In achieving elimination of higher molecular weight impurities, it is desirable to achieve recoveries of thrombin of at least 70%, at least 75%, at least 80%, at least 85%, at least 90%, at least 95%, or at least 99%.

In a preferred embodiment, the thrombin is substantially free of impurities having a molecular weight of greater than 40 kDa. In another preferred embodiment, the thrombin is substantially free of impurities having a molecular weight in the range of 40 kDa to 300 kDa. Examples of high molecular weight impurities include factor Va (heavy chain (mol. wt.=105 kDa) and light chain (mol. wt.=71 kDa/74 kDa)) and bovine serum albumin (BSA; mol. wt.=66 kDa). Viral particle impurities are also examples of high molecular weight impurities.

In a specific embodiment, the invention provides thrombin compositions substantially free of Factor Va. In some embodiments, factor Va is reduced by at least 50%, at least 55%, at least 60%, at least 65%, at least 70%, at least 75%, at least 80%, at least 85%, at least 90%, at least 95%, or at least 99%. Preferably, the absence or reduced levels of factor Va is determined by routine methods known in the art, e.g., chromatographic methods, including gel electrophoresis, factor Va activity assays and antibody based assays. In other embodiments, the amount of factor Va is reduced to less than 0.4, less than 0.35, less than 0.3, less than 0.25, less than 0.2, less than 0.15, less than 0.1, less than 0.02 μg/1000 units of thrombin or any other currently undetectable amount.

In another specific embodiment, the invention provides thrombin compositions substantially free of viral particle impurities. Viruses that can be removed by the methods of the present invention include, but are not limited to, bovine viral diarrhea virus (BVDV), pseudorabies virus (PRV), encephalomyocarditis virus (EMCV), bovine parvovirus (BPV), canine parvovirus (CPV), stickleback virus (SBV), tick-borne encephalitis virus (TBEV), equine rhinovirus 1 (ERV-1), human immunodeficiency virus 1 (HIV-1), hepatitis A (HAV), hepatitis B (HBV), and hepatitis C(HCV). Viruses can be detected by a variety of antibody based assays, including ELISAs and nucleic acid based assays, including PCR and hybridization assays. Methods for purifying thrombin to eliminate at least 50%, at least 55%, at least 60%, at least 65%, at least 70%, at least 75%, at least 80%, at least 85%, at least 90%, at least 95% and at least 99% of viral agents are provided. The invention also encompasses thrombin compositions substantially free of viral agents. In certain embodiments, viral clearance is at a log reduction value (LRV) greater than 3.5, preferably greater than 4.0, more preferably greater than 4.5. In certain embodiments, prion clearance is at a log reduction value (LRV) greater than 3.5, preferably greater than 4.0, more preferably greater than 4.5.

Thrombin purified by the methods of the present invention can be formulated for clinical use. A formulation as the term is used herein means a selection of chemical components which are mixed in order to provide advantageous properties to the final mixture.

The present invention solves many of the problems of prior art thrombin formulations. The higher purity thrombin produced has an enhanced specific activity, and reduces adverse reactions in patients who use bovine thrombin due to the elimination of high molecular weight impurities, such as factor Va.

### 6. EXAMPLES

### Example 1

### Preparation of Pre-Purified Thrombin

Pre-Purified or Low Purity Bovine Thrombin

A pre-purified bovine thrombin formulation suitable for use in this invention can be prepared as follows.

Fresh Bovine Lung is ground in conventional grinding equipment. Ground Bovine Lung may be used immediately or stored frozen in poly-lined containers at <−15° C. The ground lung is suspended in dilute sodium chloride at about 0-15° C. and extracted for about 12-72 hours. The lung suspension is filtered through coarse fabric and/or, alternatively, by centrifugation, and the liquid extract is collected.

While under agitation, approximately 100 ml of an approximately 50% suspension of magnesium hydroxide gel is added per liter of lung extract and thoroughly mixed. The suspension is centrifuged or, alternatively, filtered with filter aids and the centrifugate or filtrate collected. The adsorbed lung extract is fractionated by adding, under agitation at about 0-15° C., approximately one liter of cold saturated ammonium sulfate per liter of lung extract and mixed for about 15-480 minutes. The insoluble paste is harvested by centrifugation or, alternatively, by filtration with filter aids.

The paste is resolubilized in about 0.25 to 1 liter of cold dilute sodium chloride per liter of starting lung extract. The fractionated lung extract is reprecipitated by adding under agitation at about 0-15° C., approximately one liter of cold saturated ammonium sulfate per liter of solution, and mixed for about 15-480 minutes. The insoluble paste is harvested by centrifugation or, alternatively, by filtration with filter aids. The second paste is resolubilized in cold dilute sodium chloride and clarified by filtration.

The thromboplastin solution is concentrated in a suitable ultrafilter system to about 10-50% of the original volume and then diafiltered to remove detectable ammonium sulfate. Ultrafiltration is a process whereby a solution with a solute of molecular size that is much greater than that of the solvent is separated from the solvent by the application of hydraulic pressure. The hydraulic pressure forces the solvent through a suitable membrane and concentrates the solute.

The diafiltration is conducted by adding 8 or more volumes of 0.05M NaCl or until the permeate passed the barium chloride test. Diafiltration is a process of separating microsolutes from a solution of larger molecules by ultrafiltration with a continuous addition of solvent. The concentrate is then further optionally concentrated and the ultrafiltration completed. The ultrafiltration system is rinsed with several liters of chilled dilute sodium chloride and this wash is added to the concentrate. The pH of the concentrate is adjusted to about 7.0 with dilute hydrochloric acid or dilute sodium hydroxide. The resulting Thromboplastin is stored at about −15° C. or colder in sealed plastic containers.

Fresh Bovine Plasma, citrated, is received either frozen or chilled in a tank truck. If received frozen, the plasma is generally stored frozen until thawed for usage. Thawed plasma is maintained at 0-10° C. in stainless steel tanks. The pH of the plasma is adjusted with buffered acetic acid to about 6.6-6.8 and held for about 3-30 hours. At the end of the hold time, the Plasma is clarified. The clarified plasma is adjusted with sodium hydroxide solution to about pH 6.9-7.2.

Purification of Bovine Thrombin

Under agitation and at a temperature of about 0-10° C., about 1.5-2.5 (dry weight) grams of ion exchange resin are added per liter of Bovine Plasma and mixed 0.5-6 hours while controlling the pH at about 6.9-7.2. The resulting suspension is filtered or centrifuged to harvest the resin. The resin is washed thoroughly with 0.15-0.2 molar phosphate buffered saline at about pH 6.9-7.2 and saved.

Prothrombin is eluted from the washed resin using 0.5-1 molar phosphate buffered saline at a pH of approximately 6.9-7.2, filtered, and the extracts saved and pooled for further processing. Resins that can be used include, but are not limited to, DEAE-Sephadex A-50, Macro-Prep DEAE Support, Macro-Prep High Q Support, Macro-Prep Q Support, UNOsphere Q ion exchange, Capto Q, DEAE-Sepharose Fast Flow, Q Sepharose™ HP or equivalent. The combined extracts may be coarse filtered if desired prior to ultrafiltration. The spent resin may be treated with acid and stored prior to subsequent regeneration and reuse in a similar prothrombin complex manufacturing process.

The Prothrombin Complex extract is concentrated in a suitable ultrafilter system to about 10-50% of the original volume and then diafiltered to remove unwanted salts. The diafiltration is first conducted by adding approximately two to five liters of chilled purified water per liter of concentrate as permeate is removed, and then by adding approximately two to five liters of chilled dilute sodium chloride per liter of concentrate as permeate is removed. The concentrate is then further optionally concentrated and the ultrafiltration completed. The ultrafiltration system is rinsed with several liters of chilled dilute sodium chloride and this wash is added to the concentrate. The resulting Prothrombin Complex is stored at about −15° C. or colder in sealed containers.

Prothrombin Complex is thawed at about 35° C. or less. Prothrombin Complex is diluted to approximately 1,000-5,000 u/ml by addition of purified water containing sufficient calcium chloride to make the final calcium chloride concentration about 0.005-0.03 molar. Thromboplastin suspension is added concurrently to the prothrombin complex with the calcium chloride, under gentle agitation. The pH is adjusted to about 7.3 and mixed for about 15-60 minutes. Following activation at about 15-30° C., the suspension is chilled to about ≦10° C.

The activated Prothrombin Complex is diluted to approximately 500-3,000 u/ml with dilute sodium citrate buffer, pH about 6.6. The material may be refiltered as needed.

The pH of the above described mixture is adjusted to about 6.6 by the addition of dilute hydrochloric acid or dilute sodium hydroxide. The activated Prothrombin Complex is added to a cation exchange resin which has been adjusted to a pH of about 6.6. Resins that can be used include, but are not limited to, Amberlite CG-50, Macro-Prep CM Support, Macro-Prep High S Support, Macro-Prep S Support, UNOsphere S ion exchange, SP Sepharose™ HP or equivalent.

The column is washed with dilute sodium citrate pH 6.6 and then washed with about 0.1 to 0.25 molar sodium chloride to remove low affinity proteins which are discarded. This is followed by application of approximately 0.5-1 molar sodium chloride to elute the purified thrombin. The eluate is collected in fractions which are combined according to an in-process assay. The non-sterile bulk may be stored at about 0-10° C. for up to about 48 hours while in-process. The nonsterile bulk thrombin is formulated to no less than about 1000 u/mL by addition of water for irrigation, approximately 0.8% mannitol and approximately 0.15-0.3M sodium chloride. The pH of the formulated thrombin solution is adjusted to pH of about 6.7±1.0 with dilute hydrochloric acid or sodium hydroxide. The formulated non-sterile bulk thrombin may be stored at about 0-10° C. for up to approximately 48 hours prior to sterile processing.

The formulated non-sterile bulk thrombin is sterilized by passage through sterile, bacterial retentive non-fiber releasing filters into a suitable sterile holding tank. The resulting product is a highly concentrated α-thrombin which has a MW of about 40 kDa. Samples have an average specific activity of ≧about 1500 u/mg of protein.

In addition, prion clearance studies were performed using an ion exchange chromatographic purification step. The results indicate that the prion clearance level obtained by the thrombin chromatographic purification step is equal to 3.5 logs.

The small-scale column used had an inner diameter of 1.6 cm and was packed to a height of 50.2 cm with resin. So the column bed volume was equal to about 101 mL. The packed column was equilibrated with 100 mL of 1M NaCl followed by 100 mL of 0.025M Na-citrate pH 6.61. The flow rate of the chromatography system was set at 3.3 mL/min.

The spike sample consisted of 8 mL of 263K Strain Scrapie Hamster Brain Homogenate. The homogenate was sonicated for 20 minutes, then filtered through a 0.45, 0.2, and 0.1 μm filters. After sample spike, a total of 12 mL was taken for the pre-chromatography tests leaving a pre-column spiked sample of 396 mL (400+8−12 mL).

The pre-equilibrated column was loaded with the 396 mL of spiked crude thrombin, washed with 144 mL of 0.025M Na-citrate buffer until eluent absorbance was below 0.4AU, and washed with 275 mL of 0.2M NaCl until eluent absorbance was below 0.2AU. The column was then stripped with 0.65M NaCl and 37 mL of purified thrombin was collected from the time the absorbance reached 2AU until the time it fell back to 2AU.

The pre- and post chromatography samples collected were stored at −60° C. or below prior to performing the prion Western Blot assay. Results are shown in Table 1.

TABLE 1

| | | | | | Log |
|---|---|---|---|---|---|
| Sample | Sample | Sample | Final Titer | Total log₁₀ | Reduction |
| Record Code | Description | volume (mL) | (log₁₀(PrP^{RES}/mL)) | (PrP^{RES})* | Value |
| 1 | Spiked Load | 396 | 5.8 | 8.4 | 3.5 |
| 2 | Post column | 37 | 3.3 | 4.9 | |

*Total log₁₀(PrP^{RES}) = Final Titer (log₁₀(PrP^{RES}/mL)) + log₁₀(Sample volume (mL))

### Example 2

### Viral Clearance Using Size Exclusion Filtration

The membranes used in this example are Omega™ 1OOK VR and are “cast from modified polyethersulfone on a highly porous polyolefin backing that imparts strength and rigidity to the finished membrane.” The theoretical molecular weight cut off point is 100 kDa. Passage of small molecules is possible only under tangential flow filtration conditions. Large molecules and viruses are retained by size exclusion. The log reduction value (LRV) for bovine Parvovirus (BPV), which is a very small (20 nm) non-enveloped virus, has been determined to exceed about 3.5 logs.

The thrombin solution evaluated during this viral clearance study is a pre-purified thrombin. Samples typically have a specific activity of greater than about 1500 u/mg of protein. The protein concentration is estimated at approximately 1.2% and the salt concentration at about 0.65M NaCl. The major component of this thrombin solution is the Active Pharmaceutical Ingredient α-thrombin, which has a molecular weight of approximately 40 kDa.

There are several considerations when choosing a panel of model viruses to be included in a viral clearance study. One is to model relevant viruses that have a clear potential of contaminating the starting materials. Another is to include viruses that have a broad range of physical and chemical characteristics, in the panel of model viruses, so that if the virus clearance study shows good clearance of these viruses, then there is assurance that the manufacturing procedure can effectively clear unexpected viral agents.

It is important to consider Bovine Parvo Virus (BPV) because it is a relevant virus that has a clear potential of contaminating the starting materials, and also is extremely small, non-enveloped, and very resistant to physico-chemical treatments. Xenotropic Murine Leukemia Virus (XMuLV), Bovine Viral Diarrhea Virus (BVDV), and Pseudorabies Virus (PRV) are also included as well as BPV. This panel of viruses provides model viruses for the relevant viruses, and provide a good range of physical and chemical characteristics such that clearance of these viruses would suggest that the manufacturing procedure could clear the unexpected agents. The characteristics of the panel of viruses are indicated in Table 2.

TABLE 2
CHARACTERISTIC SUMMARY OF THE FOUR VIRUSES CHOSEN.

| | | | | | Resistance to |
|---|---|---|---|---|---|
| | | | | | physico- |
| Virus | Genome | Envelope | family | Size (nm) | chemical agents |
| BPV | DNA | No | Parvo | 20-25 | High |
| XMuLV | RNA | Yes | Retro | 80-110 | Low |
| PRV | DNA | Yes | Herpes | 150-200 | Medium |
| BVDV | RNA | Yes | Flavi | 40-70 | Medium |

For each of the four viruses considered, two filtration runs are performed: one at a target feed pressure of 8 psi, and the other at a target feed pressure of 12 psi. Each run is performed using a new Omega™ 100K VR membrane.

All runs are performed in a cold room. Each run consists of spiking the sample with 5%, (v/v) of one of the four viruses, filtering through a 0.45 μm filter to remove any virus aggregates, then filtering through the Pall Omega 100K VR membrane. Virus testing is performed on samples taken post spike, post 0.45 μm filtration, and post Omega 100K VR membrane filtration.

Thrombin filtration consists of filtering about 400 mL of pre-purified thrombin through a 0.1 ft² Omega 100K VR membrane. After 80% (320 mL) of the initial thrombin volume is collected in the permeate, the remaining 80 mL retentate still contains a lot of thrombin in addition to viruses and non-thrombin impurities. Continuous diafiltration of this 80 mL solution is used in order to maximize thrombin transmittance. This is achieved by diafiltering the 80 mL with 6× that volume (480 mL) with a NaCl solution.

The final permeate volume is thus twice the volume of the initial thrombin sample. Concentration of this permeate through a 10K VR membrane cassette is then performed to bring back the volume and concentration to the desired level. The purity of the final product is much enhanced as a result of this filtration. For example, the specific activity is increased by more than 30% and the factor Va content is reduced to undetectable levels in the final product as measured by competitive enzyme-linked immunosorbant assay (cELISA).

Virus removal (and large molecule removal) occurs by size exclusion. Very high log reduction values are observed for the 4-virus panel considered (Bovine Parvo Virus, Bovine Viral Diarrhea Virus, Xenotropic Murine Leukemia Virus, and Bovine Pseudorabies Virus). Stability of the post-Omega thrombin product is not compromised due to the increased product purity.

Table 3 summarizes the parameters and conditions of 8 filtration runs. Since the pre-filtration thrombin sample is equal to 400 mL and the filter surface area is equal to 0.1 ft², the ratio of thrombin volume to filter surface area is 4 L/ft². The volume of the virus spike is equal to 20 mL per run (5% v/v). The feed pressure is maintained at 8±2 psi for the first run and 12±2 psi for the second. The retentate pressure is equal to 0±2 psi for all runs.

TABLE 3
SUMMARY OF THE PARAMETERS AND CONDITIONS OF THE 8
FILTRATION RUNS.

| | Virus | | | | | | | |
|---|---|---|---|---|---|---|---|---|
| | PRV | | BVD | | BPV | | XmuLV | |
| | Run# | | | | | | | |
| | 1 | 2 | 1 | 2 | 1 | 2 | 1 | 2 |
| Filter surface area (ft) | 0.1 | 0.1 | 0.1 | 0.1 | 0.1 | 0.1 | 0.1 | 0.1 |
| Volume of initial thrombin (mL) | 400 | 400 | 400 | 400 | 400 | 400 | 400 | 400 |
| Volume of initial spiked thrombin (mL) | 420 | 420 | 420 | 420 | 420 | 420 | 420 | 420 |
| Volume of post filtration thrombin (mL) | 820 | 820 | 820 | 820 | 820 | 820 | 820 | 820 |
| Initial cross flow (mL/min) | 41 | 54 | 43 | 54 | 44 | 56 | 43 | 56 |
| Final cross flow at end of filtration (mL/min) | 38 | 51.5 | 40 | 50 | 41 | 55 | 40 | 53.5 |
| Feed Pressure (psi) | 8-10 | 12-14 | 8-10 | 12-14 | 8-10 | 12-14 | 8-10 | 12-14 |
| Retentate pressure (psi) | 0 | 0 | 0 | 0 | 0 | 0 | 0 | 0 |
| Total filtration time (min) | 257 | 214 | 239 | 223 | 236 | 190 | 251 | 204 |
| Pre and post use filter integrity test | Pass | Pass | Pass | Pass | Pass | Pass | Pass | Pass |

For each run, a spiked thrombin sample of 420 mL is filtered through a 0.1 ft² Omega™ 100K VR membrane. When 340 mL of permeate is collected, the remaining 80 mL of retentate solution is diafiltered with 6 times that volume using a 0.65 M NaCl solution. Therefore, the total permeate volume is equal to 340+(6×80)=820 mL. These filtration conditions yield acceptable thrombin recovery as well as enhanced degree of thrombin purity.

The cross flow at the beginning of the 8 psi runs ranges from 41-44 mL/min. Cross-flow filtration is a method of operation in which retained fluid is circulated over the membrane surface which prevent build-up of filtered material on the membrane. The cross flow at the beginning of the 12 psi runs ranges from 54-56 mL/min. The process time for the 8 psi runs ranges from 236-257 min. The process time for the 12 psi runs ranges from 190-223 min. The clearance results of the four different viruses are summarized in Table 4.

TABLE 4
SUMMARY OF VIRAL CLEARANCE RESULTS.

| | Virus | | | | | | | |
|---|---|---|---|---|---|---|---|---|
| | PRV | | BVD | | BPV | | XMuLV | |
| | Units | | | | | | | |
| | Titer + 95% CI | | Titer + 95% CI | | Titer + 95% CI | | Titer + 95% CI | |
| | (Log₁₀PFU/mL) | | (Log₁₀TCID₅₀/mL) | | (Log₁₀TCID₅₀/mL) | | (Log₁₀TCID₅₀/mL) | |
| | Run | | | | | | | |
| | 1 | 2 | 1 | 2 | 1 | 2 | 1 | 2 |
| LRV per | ≧4.92 | ≧4.92 | 4.35 | 4.22 | 3.83 | 3.62 | 3.86 | 4.47 |
| run | | | | | | | | |

| | | | | |
|---|---|---|---|---|
| Average | ≧4.92 | ≧4.29 | ≧3.74 | ≧4.26 |
| LRV per | | | | |
| Virus | | | | |

The high clearance values achieved and the similarity of the results obtained between the duplicate runs for all of the viruses indicate that the filtration step is robust. The average log reduction values were above 4 for all of the viruses except BPV, which had a log reduction value of 3.74±0.39. Even though this log reduction value was slightly below 4 logs, it is still very high under the set of conditions used.

In addition, prion clearance studies were performed using the size exclusion filtration step. The results indicate that the prion clearance level obtained by the thrombin filtration purification step is equal to 3.6 logs.

The volume of the pre-spike thrombin sample was equal to 400 mL.

The spike sample consisted of 8 mL of 263K Strain Scrapie Hamster Brain Homogenate. The homogenate was sonicated for 20 minutes, then filtered through a 0.45, 0.2, and 0.1 μm filters.

After sample spike, 12 mL were taken for the pre-Omega filtration tests leaving a pre-filtration spiked sample of 396 mL (400+8−12 mL).

The filter used was Pall's Omega™ 100K VR membrane with a surface area of 0.1 ft². So the ratio of thrombin volume to filter surface area was 4 L/ft².

The Omega™ 100K VR membrane was set up on the filtration system and rinsed with 500 mL of purified water. The pre-use integrity test was performed and passed the acceptance criteria. The membrane was then conditioned with 100 mL of 0.65M NaCl at a feed pressure of 10 psi. Permeate and cross flow rates, measured in graduated cylinders, were about 5 and 52 mL/min respectively.

Filtration of the initial 396 mL of spiked sample was started. When 315 mL of filtrate (i.e. about 80% of the initial volume) was collected, the remaining sample was diafiltered with a total of 475 mL of 0.65M NaCl (i.e. about 6 times the retentate volume). The feed pressure was maintained at about 10 psi and the retentate pressure was equal to 0 psi throughout the filtration run. These filtration conditions were previously shown to yield acceptable thrombin recovery as well as high virus clearance.

The final post-filtration volume was equal to 790 mL and the process time was equal to 172 minutes.

Pre- and post Omega filtration samples collected were stored at −60° C. or below prior to performing the prion Western Blot assay. Results are summarized in Table 5.

TABLE 5

| | | | | | Log |
|---|---|---|---|---|---|
| Sample | Sample | Sample | Final Titer | Total log₁₀ | Reduction |
| Record Code | Description | volume (mL) | (log₁₀(PrP^{RES}/mL)) | (PrP^{RES})* | Value |
| 1 | Spiked Load | 396 | 5.7 | 8.3 | 3.6 |
| 2 | Post Omega | 790 | 1.8 | 4.7 | |

*Total log₁₀(PrP^{RES}) = Final Titer (log₁₀(PrP^{RES}/mL)) + log₁₀(Sample volume (mL))

### Example 3

### Viral Clearance Using Ion Filters

Purified thrombin is first concentrated using a Pall filter with 10K MWCO and 1 ft² surface area. Then, the concentrated samples are diluted with purified water to the desired salt concentration. A total of 15 batches are prepared. The results of all the batches prepared are summarized in Tables 6 and 7.

TABLE 6
AVERAGE PERCENT RECOVERY FOR VARIOUS RUNS.

| | | % | Log Reduction for |
|---|---|---|---|
| Run # | Description | Recovery | BPV |
| 1, 2, & 3 | 50 mM NaCl, 0.8% | 74 | ≧5.12 ± 0.24 |
| | Mannitol, pH ˜5.5 | | |
| 11, 12, & | 50 mM NaCl, 0.8% | 76.9 | NA |
| 13 | Mannitol, pH ˜7.0 | | |
| 8, 9, & 10 | 72 mM NaCl, 0.8% | 90.8 | NA |
| | Mannitol, pH ˜7.0 | | |
| 14, 15, & | 72 mM NaCl, no | 91.7 | 3.60 ± 0.62 |
| 16 | Mannitol, pH ˜7.0 | | |
| 4, 5, & 7 | 108 mM NaCl, 0.8% | 99.8 | 1.25 ± 0.55 |
| | Mannitol, pH ˜7.0 | | |

TABLE 7
SUMMARY OF RESULTS.

| | Run# | | | | | | | | |
|---|---|---|---|---|---|---|---|---|---|
| | 1 | 2 | 3 | 4 | 5 | 6 | 7 | 8 | 9 |
| NaCl concentration | 50 | 50 | 50 | 108 | 108 | 81 | 108 | 72 | 72 |
| (mM) | | | | | | | | | |
| Initial Potency | 30192 | 21332 | 22111 | 32660 | 32660 | 32660 | 27217 | 24107 | 23950 |
| (u/mL) | | | | | | | | | |
| Initial Volume (mL) | 600 | 500 | 500 | 400 | 400 | 400 | 400 | 290 | 300 |
| Concentration to | 213 | 175 | 151 | 250 | 350 | 350 | 310 | 150 | 155 |
| (mL) | | | | | | | | | |
| Dilution with H₂O | 1:13 | 1:13 | 1:13 | 1:6 | 1:6 | 1:8 | 1:6 | 1:9 | 1:9 |
| Final volume of | 2720 | 2245 | 1930 | 1500 | 2100 | 2800 | 1860 | 1350 | 1395 |
| formulated sample | | | | | | | | | |
| (mL) | | | | | | | | | |
| Final potency of | 4650 | 3753 | 4201 | 8562 | 6594 | 4135 | 5559 | 4719 | 4768 |
| formulated sample | | | | | | | | | |
| (u/mL) | | | | | | | | | |
| % Recovery | 70 | 79 | 73 | 98.3 | 106 | 88.6 | 95 | 91.1 | 92.6 |
| Mannitol (% w/v) | 0.8 | 0.8 | 0.8 | 0.8 | 0.8 | 0.8 | 0.8 | 0.8 | 0.8 |
| pH | 5.49 | 5.51 | 5.53 | 7.04 | 7.05 | 7.02 | 7.03 | 7.10 | 7.05 |

| | Run# | | | | | | |
|---|---|---|---|---|---|---|---|
| | 10 | 11 | 12 | 13 | 14 | 15 | 16 |
| NaCl concentration | 72 | 50 | 50 | 50 | 72 | 72 | 72 |
| (mM) | | | | | | | |
| Initial Potency | 29161 | 22928 | 23836 | 24063 | 26569 | 22474 | 23977 |
| (u/mL) | | | | | | | |
| Initial Volume (mL) | 300 | 300 | 300 | 300 | 300 | 300 | 300 |
| Concentration to | 145 | 117 | 105 | 115 | 140 | 123 | 125 |
| (mL) | | | | | | | |
| Dilution with H₂O | 1:9 | 1:13 | 1:13 | 1:13 | 1:9 | 1:9 | 1:9 |
| Final volume of | 1305 | 1521 | 1365 | 1495 | 1260 | 1107 | 1125 |
| formulated sample | | | | | | | |
| (mL) | | | | | | | |
| Final potency of | 5954 | 3288 | 4534 | 3454 | 5806 | 5781 | 5658 |
| formulated sample | | | | | | | |
| (u/mL) | | | | | | | |
| % Recovery | 88.8 | 72.7 | 86.5 | 71.5 | 91.8 | 94.9 | 88.5 |
| Mannitol (% w/v) | 0.8 | 0.8 | 0.8 | 0.8 | 0 | 0 | 0 |
| pH | 7.05 | 7.05 | 7.07 | 7.02 | 6.98 | 6.95 | 7.03 |

Samples from run 1-3 (50 mM NaCl) are initially used for viral clearance validation of the Mustang Q filter and result in very high log reduction value for Bovine Parvo Virus (BPV). However, since the thrombin recovery is low, averaging only 74%, the viral clearance study is repeated using samples from run 4, 5, and 7 (108 mM NaCl), which yield an average thrombin recovery of 99.8% but less than 2 log reduction values for BPV. Finally, the viral clearance study is repeated using samples from run 14, 15, and 16 (72 mM NaCl and 91.7% recovery) and the log reduction value for BPV was acceptable (3.6±0.62).

In addition prion reduction studies were done using an ion exchange filter. The results indicate that the prion clearance level obtained by the thrombin filtraton purification step is equal to greater than 3.9 logs.

The volume of the pre-spike thrombin sample was equal to 91 mL.

The spike sample consisted of 1.6 mL of 263K Strain Scrapie Hamster Brain Homogenate. The homogenate was sonicated for 20 minutes, then filtered through a 0.45, 0.2, and 0.1 μm filters.

12 mL were taken for the pre-Mustang Q filtration tests leaving a pre-filtration spiked sample of 80.6 mL (91+1.6−12 mL).

The filter used was Pall's Mustang Q filter with a surface area of 0.35 mL. So the ratio of thrombin volume to filter surface area was 230 mL of sample per mL of filter.

The filter holder was sanitized without filter coin with 20 mL of 1N NaOH with a 20 min hold. The Mustang Q filter was placed in the holder, washed with 20 mL of 1N NaOH followed with 1M NaCl wash until eluent pH was neutral.

Then the filter was conditioned with 25 mL of 72 mM NaCl at a flow rate of about 3 mL/min before the actual filtration of the 80.6 mL spiked sample.

The final post-filtration volume was equal to 77 mL and the process time was equal to 49 minutes.

Pre- and post ion filtration samples collected were stored at −60° C. or below prior to performing the prion Western Blot assay. The results are shown in Table 8.

TABLE 8

| | | | | | Log |
|---|---|---|---|---|---|
| Sample | Sample | Sample | Final Titer | Total log₁₀ | Reduction |
| Record Code | Description | volume (mL) | (log₁₀(PrP^{RES}/mL)) | (PrP^{RES})* | Value |
| 1 | Spiked Load | 80.6 | 4.8 | 6.7 | >3.9 |
| 2 | Output | 77 | <0.9 | <2.8 | |

### Example 4

### Size Exclusion Filtration Under Various Conditions

This example also uses size exclusion filtration using Pall Omega 100K VR filters. Three runs are performed at a feed pressure of 8 psi and three are performed at a feed pressure of 12 psi. The parameters of the scaled-down filter are chosen to keep the volume to filter surface area constant, and assure operation in the specified feed pressure range.

Each run is performed with a new 0.1 ft² Pall Omega 100K VR filter and all runs are performed in a cold room (≦about 8° C.). A flow meter is included in the system to better monitor the cross-flow during filtration. The flow meter is calibrated in the cold room prior to use.

Table 9 summarizes the conditions and parameters of the six filtration runs. For the 8 psi runs, thrombin activity of the starting material averages 22,091 u/mL and for the final filtrate pool, it averages 11,130 u/mL. The resulting percent of thrombin recovery after 6 diafiltration runs cycles averages 86%. Runs performed at a feed pressure of 8 psi show slightly more thrombin recovery than at 12 psi.

TABLE 9
SUMMARY OF THROMBIN FILTRATION AND RECOVERY
RESULTS

| | Run # 1 | Run # 2 | Run # 3 | Run # 4 | Run # 5 | Run # 6 |
|---|---|---|---|---|---|---|
| Target Feed | 8 | 8 | 8 | 12 | 12 | 12 |
| Pressure | | | | | | |
| Thrombin | 400 | 400 | 400 | 400 | 400 | 400 |
| Volume (mL) | | | | | | |
| Spike Volume | 20 | 20 | 20 | NA | NA | NA |
| (mL) | | | | | | |
| Total volume | 420 | 420 | 420 | 400 | 400 | 400 |
| Pre-filtration | | | | | | |
| sample (mL) | | | | | | |
| Activity Pre- | 22,696 | 24,177 | 19,399 | 21,559 | 21,559 | 20,747 |
| filtration sample | | | | | | |
| (mL) | | | | | | |
| Total activity Pre- | 9,532,320 | 10,154,340 | 8,147,580 | 8,623,600 | 8,623,600 | 8,298,800 |
| filtration sample | | | | | | |
| (u) | | | | | | |
| Total volume | 820 | 820 | 820 | 800 | 800 | 800 |
| Post-filtration | | | | | | |
| sample (mL) | | | | | | |
| Activity Post- | 10,405 | 13,210 | 9,776 | 8,631 | 9,940 | 8,923 |
| Filtration sample | | | | | | |
| (u/mL) | | | | | | |
| Total activity Post | 8,532,100 | 10,832,200 | 8,016,320 | 6,904,800 | 7,952,000 | 7,138,400 |
| filtration sample | | | | | | |
| (u) | | | | | | |
| % thrombin | 89.5 | 106.7 | 98.4 | 80.0 | 92 | 86 |
| recovery | | | | | | |
| Total process | 206 | 219 | 215 | 155 | 173.5 | 166 |
| time | | | | | | |

One difference is that at a feed pressure of 12 psi, the cross flow is higher resulting in a faster passage of thrombin, thereby shortening the processing time.

Table 10 shows that the filtration step results in a 36.4% increase in thrombin purity or specific activity for the 8 psi runs, and a 37.1% increase for the 12 psi runs. The specific activity increases from a range of 1688.4 to 1986.0 of thrombin/mg protein in the prefiltration samples to a range of 2324.6 and 2690.1 of thrombin/mg protein in the post filtration samples.

TABLE 10
SPECIFIC ACTIVITY OF PRE- VS. POST-OMEGA 100 FILTRATION
SAMPLES

| | | Run # 1 | Run # 2 | Run # 3 | Avg | Run # 4 | Run # 5 | Run # 6 | Avg |
|---|---|---|---|---|---|---|---|---|---|
| Target feed | | 8 | 8 | 8 | 8 | 12 | 12 | 12 | 12 |
| pressure (psi) | | | | | | | | | |
| Pre- | Activity | 22,696 | 24,177 | 19,399 | | 21,559 | 21,559 | 20,747 | |
| Omega | (u/mL) | | | | | | | | |
| 100 | Protein | 13.056 | 12.576 | 11.1406 | | 10.8554 | 11.7673 | 12.2881 | |
| Filter | (mg/mL) | | | | | | | | |
| | Specific | 1738.4 | 1922.5 | 1741.3 | 1800.7 | 1986.0 | 1832.1 | 1688.4 | 1835.5 |
| | Activity | | | | | | | | |
| | (u/mg) | | | | | | | | |
| Post | Activity | 10,405 | 13,210 | 9,776 | | 8,631 | 9,940 | 8,923 | |
| Omega | (u/mL) | | | | | | | | |
| 100 | Protein | 4.236 | 5.108 | 4.2055 | | 3.5337 | 3.695 | 3.7365 | |
| Filter | (mg/ml) | | | | | | | | |
| | Specific | 2456.3 | 2586.1 | 2324.6 | 2455.7 | 2,442.5 | 2,690.1 | 2,338.1 | 2490.2 |
| | Activity | | | | | | | | |
| | (u/mg) | | | | | | | | |

| | | | | | | | | |
|---|---|---|---|---|---|---|---|---|
| % increase in | 41.3 | 34.5 | 33.5 | 36.4 | 23.0 | 46.8 | 41.4 | 37.1 |
| Specific Activity | | | | | | | | |
| due to | | | | | | | | |
| nanofiltration | | | | | | | | |

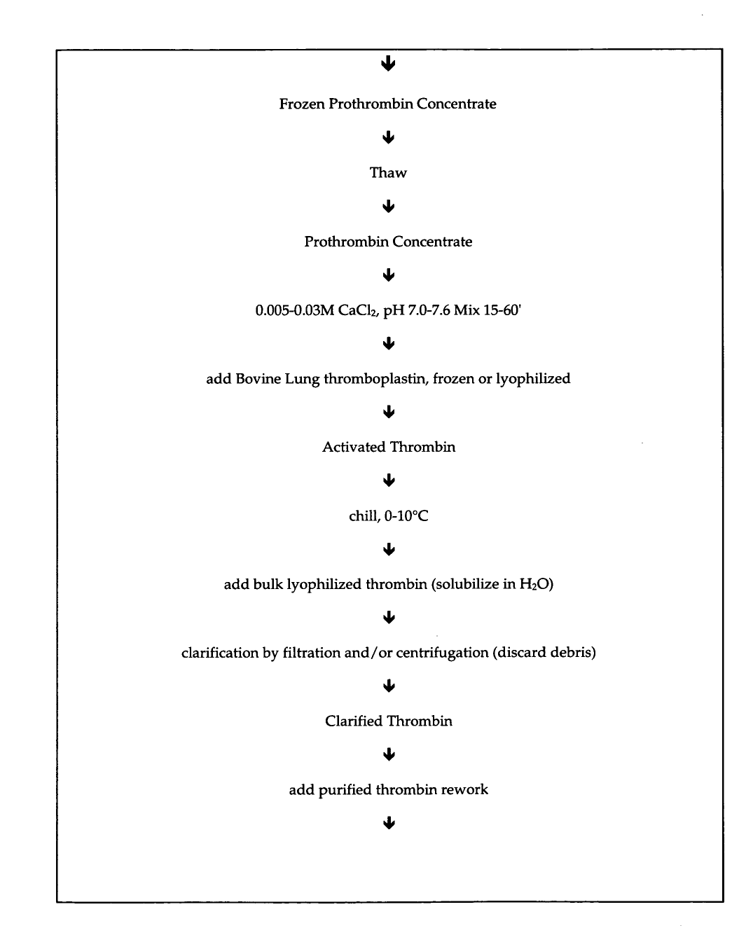

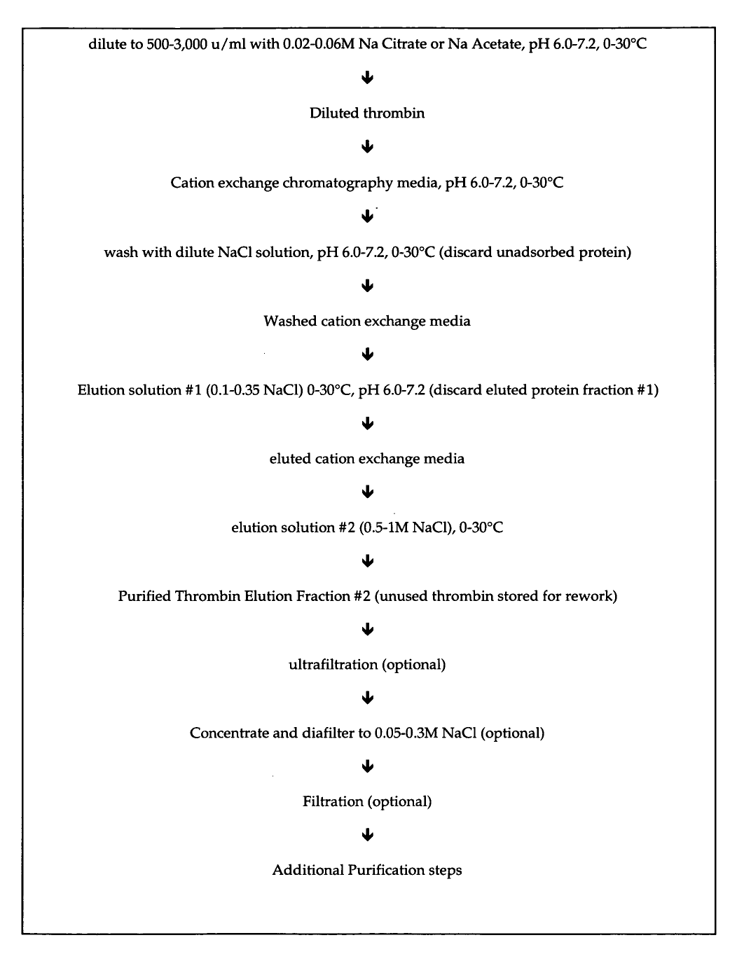

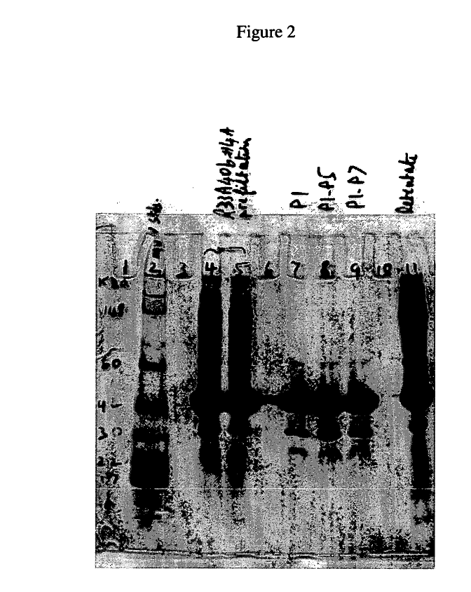

The permeate fractions are much cleaner than the respective initial starting thrombin sample as shown in FIG. 2. Almost all of the high molecular weight impurities observed in the starting material are retained by the filter in the retentate.

Table 11 shows that the filtration step also results in a substantial reduction in Factor Va content. The average reduction between the runs performed at the two feed pressures is comparable: 88.5% in 8 psi runs, and 89.3% in the 12 psi runs. Factor V/Va is associated with coagulopathies that may occur in patients in response to surgical exposure to topical bovine thrombin. Current knowledge suggests that factor V/Va contamination of bovine thrombin stimulates the production of patient antibovine Factor Va antibodies which can cross-react with the patient's own factor Va, thereby leading to impaired hemostasis. This filtration step provides the benefits of benefit of substantially reducing factor Va content to undetectable levels in the final Thrombin-JMI® as measured by competitive enzyme-linked immuno sorbant assay (ELISA).

TABLE 11
FACTOR VA CONTENT OF PRE- VS. POST-OMEGA 100
FILTRATION SAMPLES BY ELISA

| | | Run # 1 | Run # 2 | Run # 3 | Avg | Run # 4 | Run # 5 | Run # 6 | Avg |
|---|---|---|---|---|---|---|---|---|---|
| Target feed pressure (psi) | | 8 | 8 | 8 | 8 | 12 | 12 | 12 | 12 |
| Pre-Omega | (μg/mL) | 44.993 | 45.566 | 40.879 | 43.813 | 22.954 | 22.954 | 37.163 | 27.690 |
| | (μg/1000u) | 1.982 | 1.885 | 2.107 | 1.991 | 1.065 | 1.065 | 1.791 | 1.307 |
| Post-Omega | (μg/mL) | 3.625 | 2.017 | 1.801 | 2.481 | 0.741 | 0.708 | 2.357 | 1.269 |
| | (μg/1000u) | 0.348 | 0.153 | 0.184 | 0.228 | 0.086 | 0.071 | 0.264 | 0.140 |

| | | | | | | | | |
|---|---|---|---|---|---|---|---|---|
| % decrease of Factor | 82.4 | 91.9 | 91.3 | 88.5 | 91.9 | 93.3 | 85.3 | 89.3 |
| Va due to Omega | | | | | | | | |
| filter | | | | | | | | |

While specific examples have been given, these are preferred embodiments only, and are meant to further explain and describe the invention. They are not intended to define the full scope of this invention.

All references cited herein are incorporated herein by reference in their entirety and for all purposes to the same extent as if each individual publication, patent or patent application was specifically and individually indicated to be incorporated by reference in its entirety for all purposes.

What is claimed is:

1. A method for preparing a thrombin having enhanced purity, the method comprising:
(a) applying the thrombin preparation to a size exclusion filter; and
(b) excluding impurities from the thrombin preparation.

2. The method of claim 1 wherein the thrombin preparation is bovine thrombin.

3. The method of claim 1 wherein the thrombin preparation is Thrombin-JMI®.

4. The method of claim 1 wherein the size exclusion filter is capable of excluding impurities that have a molecular weight greater than 40 kDa.

5. The method of claim 1 wherein the size exclusion filter is capable of excluding impurities that have molecular weights ranging from 40 kDa to 300 kDa.

6. The method of claim 1 wherein the size exclusion filter has a molecular weight cutoff ranging from 50 kDa and 150 kDa.

7. The method of claim 1 wherein the size exclusion filter has a molecular weight cutoff is 50 kDa.

8. The method of claim 1 wherein the size exclusion filter has a molecular weight cutoff of 100 kDa.

9. The method of claim 1 wherein impurities in the thrombin preparation are reduced by at least 50%.

10. The method of claim 1 wherein impurities in the thrombin preparation are reduced by at least 80%.

11. The method of claim 1 wherein the specific activity of said thrombin preparation is increased by at least 1000%.

12. The method of claim 1 wherein the specific activity of said thrombin preparation is increased by at least 1200%.

13. The method of claim 1 wherein the specific activity of said thrombin preparation is increased by at least 1500%.

14. The method of claim 1 wherein said thrombin having enhanced purity is substantially free of impurities.

15. The method of claim 1 wherein said thrombin having enhanced purity is substantially free of factor Va.

16. The method of claim 1 wherein said thrombin having enhanced purity is substantially free of prions.

17. The method of claim 1 wherein said thrombin having enhanced purity has a prion reduction equal to at least 3.5 logs.

18. The method of claim 1 wherein said thrombin having enhanced purity is substantially free of viral agents.

19. The method of claim 1 wherein the thrombin having enhanced purity is substantially pure.

20. The method of claim 1 further comprising, applying the thrombin preparation to an ion exchange filter.

21. The method of claim 1 further comprising, applying the thrombin preparation to a chromatographic purification step.

22. The method of claim 21, wherein the chromatographic purification step comprises an ion exchange chromatography column.

23. A thrombin composition substantially free of impurities having a molecular weight greater than 40 kDa.

24. The thrombin composition of claim 23 wherein the thrombin composition is substantially free of impurities having a molecular weight between 50 kDa and 300 kDa.

25. A thrombin composition substantially free of impurities.

26. A thrombin composition that is substantially pure.

27. A thrombin composition substantially free of factor Va.

28. The thrombin composition of claim 27 wherein factor Va is measured by factor Va activity assay, ELISA, or Western Blot.

29. A thrombin composition substantially free of factor Va activity, wherein the factor Va is present at less than 0.4 μg/1000 units of thrombin.

30. A thrombin composition substantially free of viral agents.

31. A thrombin composition with a thrombin specific activity greater than 1800 u/mg of protein and substantially free of impurities having a molecular weight greater than 40 kDa.

32. A thrombin composition substantially free of viral agents, wherein the log reduction value is greater than 3.5 per virus.

33. A thrombin composition having a thrombin specific activity between about 1800 and 3000 u/mg of protein.

34. A thrombin composition of claim 33, having a thrombin specific activity between about 1800 and 2400 u/mg of protein.

35. A thrombin composition of claim 33, having a thrombin specific activity between about 2400 and 2500 u/mg of protein.

36. A thrombin composition of claim 33, having a thrombin specific activity between about 2500 and 2600 u/mg of protein.

37. A thrombin composition of claim 33, having a thrombin specific activity between about 2600 and 2700 u/mg of protein.

38. The composition of claim 31 wherein the thrombin specific activity is greater than 3000 u/mg of protein.

39. A method of preparing a thrombin having enhanced purity comprising applying a thrombin preparation to an ion exchange filter.

40. A method of preparing a thrombin having enhanced purity comprising applying a heat treatment to a thrombin preparation.

41. The method of claim 40 wherein the heat treatment includes holding the thrombin at 60° C. for 10 hours.

42. A method of preparing a thrombin having enhanced purity comprising lowering the pH below about 5 of a thrombin preparation.

43. A method of preparing a thrombin having enhanced purity comprising the application of electromagnetic radiation to a thrombin preparation.

44. The method of claim 43 wherein the electromagnetic radiation is gamma radiation.

45. The method of claim 44 wherein the electromagnetic radiation is UV radiation.

46. A method for large-scale preparation of thrombin having enhanced purity comprising applying at least 15 L of a thrombin preparation to a size exclusion filter.

47. The method of claim 46, wherein the thrombin preparation comprises 300,000,000 units of thrombin.

48. A method for preparing a thrombin having enhanced purity, the method comprising:
(a) applying the thrombin preparation to a chromatographic purification step;
(b) applying the thrombin preparation to a size exclusion filter;
(c) applying the thrombin preparation to an ion exchange filter; and
(d) excluding impurities from the thrombin preparation.

49. The method of claim 48, wherein the chromatographic purification step comprises an ion exchange chromatography column or a size exclusion chromatography column.
